(19) United States
(12) Patent Application Publication
(10) Pub. No.: US 2020/0060870 A1
(43) Pub. Date: Feb. 27, 2020

(54) Apparatus and Methods for Establishing and Maintaining Bodily Temperature Levels

(71) Applicant: University of Massachusetts, Boston, MA (US)

(72) Inventor: Amy Marie O'Connor, Dudley, MA (US)

(21) Appl. No.: 16/610,978

(22) PCT Filed: May 11, 2018
(86) PCT No.: PCT/US18/32279

§ 371 Date: Nov. 5, 2019

Related U.S. Application Data
(60) Provisional application No. 62/505,705, filed on May 12, 2017.

(51) Int. Cl. A61F 7/02

(52) U.S. Cl. CPC A61F 7/02; A61F 2007/0219; A61F 2007/0036; A61F 2007/0257; A61F 2007/0258; A61F 2007/0292

(57) ABSTRACT

The presented invention is directed to an apparatus and methods of thermal treatment for a body and/or limb of a subject. Embodiments of the invention include a composite for maintaining temperature and moisture levels surrounding the body or limb to which the composite may be applied. A preferred embodiment of the invention includes a moisture retention material, a temperature retention material, and a vapor barrier material.

### CROSS-REFERENCE TO RELATED APPLICATIONS

This application claims the benefit of U.S. Provisional Application No. 62/505,705, filed May 12, 2017, which is incorporated by reference in its entirety.

### FIELD OF INVENTION

The present invention relates generally to thermal treatment of the body. More specifically, the invention is directed to an apparatus and methods to establish and maintain a temperature level of the body or body part using a composite material.

### BACKGROUND

Homeothermic animals, such as humans and other mammals (collectively “subjects”), strive to maintain a constant internal core body temperature despite temperature fluctuations in the ambient environment. In most subjects, the thermal core generally includes the vital organs of the body, such as the brain and the several organs located within the abdomen and chest and is maintained at about 37 degrees Celsius. Peripheral tissues, such as the skin, fat, and muscles, buffer the thermal core from the external environment. Maintaining the core body temperature is vital to a healthy physical and mental condition.

From time to time though, subjects may be exposed to certain conditions that cause a change in core body temperature. For example, prolonged exposure to cold temperatures or submersion in cold water may decrease the temperature of the skin, limbs and body leading to frostbite and/or a medical condition known as hypothermia.

Frostbite is an injury that is caused by exposure of parts of the body to the cold resulting in a failure to maintain the temperature of the exposed area. This loss of temperature may damage the skin and underlying tissue. Frostbite varies in severity. In superficial frostbite—the mildest form of frostbite—damage may be limited to the uppermost layers of the skin that may be sloughed off and replaced with new skin. Deep frostbite is the most severe form of frostbite, and may result from the freezing of the skin and underlying tissue that may lead to permanent tissue loss. However, even superficial frostbite may lead to permanent tissue damage. Generally, fingers, toes, feet, and other extremities are most commonly affected by frostbite.

Hypothermia is the condition in which the core body temperature is lowered by 2 or more degrees from the average core body temperature of 37 degrees Celsius. Hypothermia is further categorized according to severity: mild hypothermia is the condition when the body core temperature is within the range of 32 degrees Celsius to 35 degrees Celsius, moderate hypothermia occurs between 30 degrees Celsius to 32 degrees Celsius, severe hyperthermia occurs between 24 degrees Celsius to 30 degrees Celsius, and profound hypothermia is the condition in which the body temperature is less than 24 degrees Celsius. Severe and profound hypothermia—which includes symptoms ranging from a decrease in heart rate, decrease in respiratory rate, decrease in blood pressure, difficulty in speaking, loss of motor functions, slurred speech, and amnesia—are the most severe types of hypothermia that may result in serious injury.

While it is unclear what is the prevalence of frostbite and hypothermia throughout the world, the United States of America reportedly averages over 500 cases of frostbite requiring emergency treatment per year and some 1300 deaths from hypothermia per year. Proper medical treatment—if provided at the earliest onset of frostbite or hypothermia symptoms—is paramount may reduce or eliminate resultant injury and death from each of these conditions.

Current medical treatment of frostbite or hypothermia may include—depending on the severity of each condition—one or more interventions all with the goal to raise the body temperature: blood rewarming in which the subject's blood has been drawn, warmed, and returned to the body; intravenous introduction of warm fluids to the subject; administering warmed air through a mask or nasal tube to the subject; or, irrigation of the subject through, for example, catheters. Each of these techniques have the shared objective of gradually warming the subject while simultaneously preventing the sudden dilation of blood vessels. Dilation may occur when heat is applied and the blood pressure to the vital organs such as the brain, heart, lungs, and kidneys decrease. Sudden dilation and reduced blood pressure may lead to cardiac arrest and death.

Conventional approaches to treating frostbite and hypothermia have a number of problems associated with them. For example, only highly skilled medical professionals are able perform the blood rewarming and the application of intravenous fluid to the subject. Further, these techniques are performed typically in hospital-like settings that provide the specialized equipment that is needed to perform the procedure, and the more sterile environment provided by the treatment center. Consequently, if the patient requires transportation to a medical facility—which may be located some distance away from the patient—critical time may be lost in treating the frostbite or hypothermia. Further, even in cases where a limb may be wrapped with a heating component, such devices quickly cool and require constant monitoring and/or replacement.

Accordingly, there is a need for an apparatus and methods by which the temperature of a subject suffering from hypothermia or frostbite may be quickly—and without special training and complicated equipment—moderated and to facilitate the gradual temperature increase of the subject or effected limb(s) over a prolonged period of time without the need for replacement or reheating/recooling of the apparatus. The present invention satisfies these needs.

### SUMMARY OF THE INVENTION

The present invention is directed to an apparatus and methods for maintaining moisture and temperature levels of the body or limb of a subject through the use of a composite material.

Generally, certain preferred embodiments of the invention may comprise a composite material including one or more of a temperature retention material, a moisture retention material, and a vapor barrier material.

In certain preferred embodiments of the invention, the temperature retention material may include a gel or gel-like material comprising water, a salt such as ammonium nitrate, ammonium chloride, magnesium sulfate or calcium chloride, polyethylene glycol, propylene glycol, a cellulose, silica, vinyl coated silica, a phase change material, or a combination of any of the above. The temperature retention material may retain a certain temperature after being exposed to a thermal source such a heating by microwave or being placed in a freezer. Alternatively, the composite may be configured to produce either an endothermic or exothermic reaction through the mixing of chemical components separately held within the temperature retention material. Separating membranes or walls within the temperature retention material (termed collectively “separating membranes”) may facilitate the separation of the chemical components. Manipulation of the composite that houses the temperature retention material may break the separating membranes to allow the mixing of the chemical components to produce the desired endothermic or exothermic reaction.

In certain embodiments of the invention, the moisture retention material may include any woven or non-woven fabric, polyester, non-woven polyester, wool, cotton, a super absorbent polymer (e.g. sodium polyacrylate and potassium polyacrylate), or a combination of any of these materials. The moisture retention material may help to maintain the temperature level of the composite for extended periods of time such as, for example, 8 to 10 hours without the need to replace the composite.

Certain embodiments of the invention also may include a vapor barrier material to insulate the composite—thereby helping to retain the temperature and moisture levels—and to prevent ambient material from contacting the body or limb, or from entering the composite. The vapor barrier material may include one or more of the following impermeable material: polyether or polyurethane laminates, vapor-retardant paints, plastics such as polyethylene, vinyl membranes, polytetraflouroethylene membranes, i.e. Goretex®, poly lactic acid, rubber material, and coated nylon. In one preferred embodiment, the vapor barrier includes one or more layers of polyethylene.

Other certain embodiments of the invention may include separate and/or additional layers of each of the temperature retention material, the moisture retention material, and the vapor barrier material. Further, some embodiment of the invention may include a semipermeable material configured to, for example, wick away perspiration from the body area to which the composite is applied.

Certain embodiments of the invention may be configured as a wearable garment, such as a glove, mitten, sock, or sleeve. Alternatively, the composite may be configured into a small thermal pack that may be directly applied to an area of the body or inserted into a wearable garment configured to receive the thermal pack and placed on the subject.

Advantageously, embodiments of the invention provide a means of treating thermal injury without the need for special training or equipment.

Advantageously, embodiments of the invention establish and maintain certain desired moisture and temperature levels surrounding a body or limb for prolonged periods of time without the need for replacement of the composite, or continuous removal and heating/cooling of the composite.

Advantageously, certain embodiments of the invention include a removable temperature retention material that may be replaced without the need to remove the entire composite from the body or limb to which it is applied. Additionally, embodiments of the invention allow for the temperature retention material to be replaced with another temperature retention material having similar or different properties. For example, thermal treatment of an injury may require alternate heating and cooling cycles. The features of certain embodiments of the invention allow the user to easily remove and replace the temperature retention material as needed.

These features and advantages of the present invention will become more fully apparent from the following description and appended claims or may be learned by the practice of the invention as described below.

### BRIEF DESCRIPTION OF THE DRAWINGS

The preferred embodiments of the invention will be described in conjunction with the appended drawings provided to illustrate and not to the limit the invention, where like designations denote like elements, and in which:

FIG. 1 illustrates a schematic view of a preferred embodiment of a composite of the invention;

FIG. 2A illustrates a schematic view of one certain embodiment of a temperature retention material having a separating membrane to separate chemical components prior to activation;

FIG. 2B illustrates a schematic view of one certain embodiment of a temperature retention material after breaking the separating membrane to activate the chemical components through mixing to produce a thermal chemical reaction;

FIG. 3 illustrates a schematic view of one certain embodiment of a composite of the invention;

FIG. 4 illustrates a schematic view of one certain embodiment of a composite of the invention; and

FIG. 5 illustrates the use of an embodiment of a composite of the invention as applied to a body or limb in need of thermal treatment.

### DETAILED DESCRIPTION OF THE INVENTION

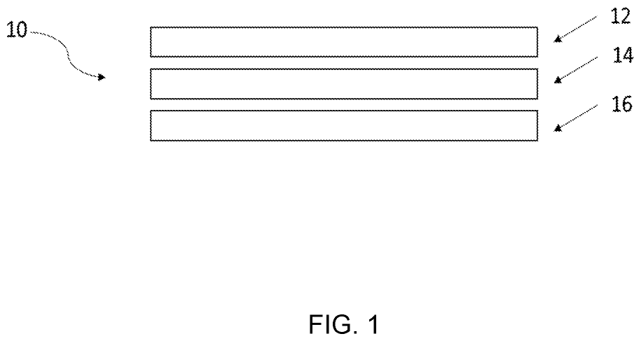

The present invention is directed to an apparatus and methods of controlling and maintaining a certain temperature and moisture level when the apparatus is applied to a body or body part to facilitate thermal treatment. FIG. 1 illustrates one certain preferred embodiment of the invention including a composite 10 of one or more layers of material that may include a temperature retention material 14, a moisture retention material 16, and vapor barrier material 12 to form a three-layered composite material 10.

The temperature retention material 14 includes any non-gaseous material that may change shape or direction in response to an external force applied to it and, further includes the capacity to retain heat or cold for transfer to the body or a body limb. The temperature retention material includes liquids, finely divided solids, gels and combinations of liquids and solid particles.

In certain preferred embodiments of the invention, the temperature retaining material 14 may be a gel or gel-like material, and more preferably a water-based gel or colloidal dispersion. For example, in some embodiments of the invention, the temperature retention material 14 may include, iron, water, activated carbon, vermiculite, salt, sodium acetate, ethylene glycol, polyethylene glycol, calcium chloride, ammonium chloride, magnesium sulfate, ammonium nitrate, urea, cellulose, hydroxyethyl cellulose, sodium carboxymethylcellulose, sodium polyacrylate, silica gel, vinyl-coated silica gel, or a combination of any of the above. In certain embodiments of the invention, the temperature retention material may be water. In one preferred embodiment of the invention, the temperature retention material may be a gel comprising about 60% to 70% water, about 20% to 35% polypropylene glycol, and less than about 5% sodium carboxymethylcellulose.

In certain embodiments of the invention, the temperature retention material 14 may include Phase change materials (PCM's) to form the gel or gel-like material. PCM's are chemical compounds that undergo a reversible phase transition at a precise temperature. Common PCM's include, for example, low molecular weight aliphatic hydrocarbons, paraffin waxes, and acids of natural oils and waxes. In particular, paraffinic hydrocarbons are well-suited for attaining a desired temperature for a given thermal therapy application due to the strong correlation between the number of carbon atoms and the melting point of the hydrocarbon. Thus, the desired therapeutic temperature may readily be attained by selection of the appropriate PCM or combination of PCM's. See for example, U.S. Pat. No. 6,689,466 incorporated by reference in its entirely. Suitable paraffinic hydrocarbons include n-Octacosane, n-Heptacosane, n-Hexacosane, n-Pentacosane, n-Tetracosane, n-Tricosane, n-Docosane, n-Hemeicosane, n-Eicosane, n-Nonadecane, n-Octadecane, n-Heptadecane, n-Hexadecane, n-Pentadecane, n-Tetradecane, and n-Tridecane. Each of these hydrocarbons display a unique phase transition temperature ranging from negative 5.5 degrees Celsius for n-Tridecane to 61.4 degrees Celsius for n-Octosane.

In certain preferred embodiments of the invention, the temperature retention material 14 may be externally heated or cooled, such as via microwave heating, freezing, or cooling in an ice bath. For example, the temperature retention material 14 may be exposed to a heat source to attain a temperature of about 37 degrees Celsius to about 42 degrees Celsius, and more preferably, about 38 degrees Celsius to about 40 degrees Celsius. Conversely, the temperature retention material may be cooled to about negative 7 degrees Celsius to about 4.5 degrees Celsius. Preferably, the temperature retention material 14 retains a certain temperature over a prolonged period of time, such as 2 to 10 hours, preferably, 5 to 10 hours, and more preferably, 8 to 10 hours to provide an uninterrupted heating or cooling of the body or limb and greatly reduces or eliminates the need to continuously change the composite.

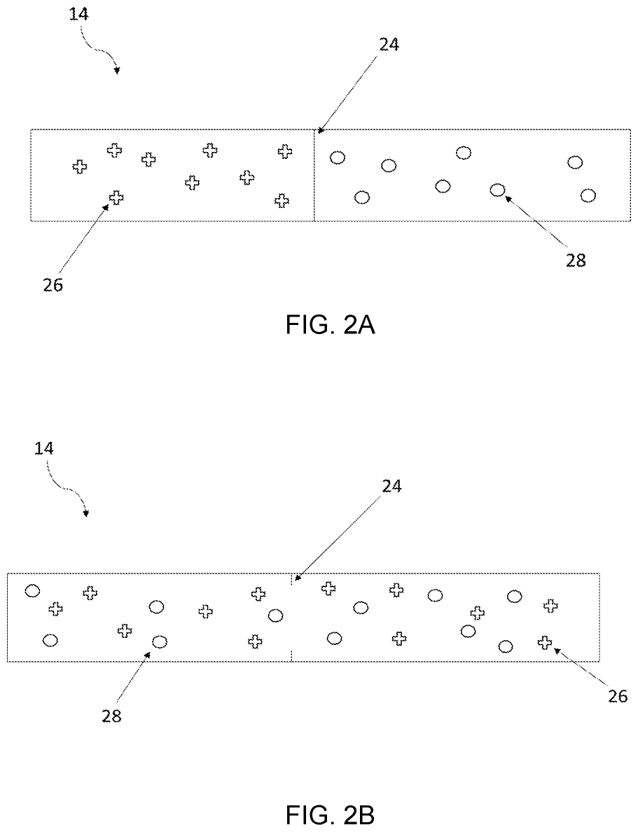

Alternatively, the temperature retention material 14 may include chemical components that, when activated, mix and react to produce an endothermic or exothermic reaction. For example, FIG. 2A illustrates the temperature retention layer 14 having one or more separating membranes 24 partitioning the one or more chemical components 26, 28 (e.g. a salt and water). As illustrated in FIG. 2B, the user may then break the separating membrane(s) 24 dividing the two or more chemical components by twisting, squeezing, or stretching the temperature retention material 14 to cause mixing of the salt and water to produce a chemical reaction that results in a sharp increase or decrease in temperature.

Certain embodiments of the invention may include a moisture retention material 16. The moisture retention material 16 may be any type of absorbent material. For example, the moisture retention material 16 may include polyester, non-woven polyester, wool, cotton, or a blend of any of these materials, saccharides, super absorbent polymer, or a combination of any one of the foregoing materials.

Superabsorbent materials suitable for use in the present invention are known to those skilled in the art, and may be in any operative form, such as a particulate form. Generally, the superabsorbent material may be a water-swellable, generally water-insoluble, hydrogel-forming polymeric absorbent material, that is capable of absorbing at least about 10, desirably about 30, and possibly about 60 times or more its weight as measured using physiological saline (e.g., 0.9 wt % NaCl). The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers also may be crosslinked to render the material substantially water insoluble. Crosslinking may, for example, be by irradiation or covalent, ionic, Van der Waals, or hydrogen bonding.

In some embodiments of the invention, the moisture retention material 16 may include a superabsorbent polymer and a nonwoven polyester web impregnated with the superabsorbent polymer, the superabsorbent polymer formed in situ, i.e., in place on the nonwoven polyester web from an aqueous superabsorbent polymer composition as disclosed, for example, in US Pat. Pub. No. 2003/0149413 incorporated by reference in its entirety.

Certain embodiments of the invention may include a vapor barrier material 12. Vapor barrier materials may be categorized according to water/moisture vapor transmission rates (Water Vapor Transmission Rates or “WVTR”) though various material according to permeability as measured in perms (1.0 US perm=1.0 grain/hour×square-foot×inHg). An impermeable vapor barrier has a perm of less than or equal to 0.1, a semi-impermeable vapor barrier has a perm of between 0.1 perm and 1.0 perm, a semi-impermeable vapor barrier has a perm of between 0.1 perm and 1.0 perm, a semi-permeable vapor barrier has a perm of between 1.0 perm and 10 perm, and permeable vapor barrier has a perm of greater than 10 perms.

A suitable technique for determining WVTR and permeance values of a material is the test procedure standardized by INDA (Association of the Nonwoven Fabrics Industry), number IST-70.4-99, entitled “Standard Test Method for Water Vapor Transmission Rate through Nonwoven and Plastic Film using a Guard Film and Vapor Pressure Sensor” which is incorporated by reference herein. The INDA procedure provides for the determination of WVTR, the permeance of the material to water vapor and, for homogeneous materials, water vapor permeability coefficient. Other known methods for determining permeance include ASTM E96 dry cup method.

The vapor barrier material 12 may be configured to keep moisture within the composite material 10 for as long as possible. The vapor barrier material 12 also may be configured to keep moisture from directly contacting the body or limb to which the composite is applied. The vapor barrier material 12 may include, for example, elastomeric coatings such as polyether or polyurethane laminates, vapor-retardant paints, plastics such as polyethylene, vinyl membranes, polytetraflouroethylene membranes, i.e. Goretex®, poly lactic acid, rubber material, and coated nylon. In one preferred embodiment of the invention, the vapor barrier material is one or more layers of polyethylene.

With reference again to FIG. 1, the vapor barrier material 12 preferably forms the outer most layer of the composite material 10 to insulate and prevent both the moisture and temperature level of the composite 10 from dissipating. Preferably, the temperature retention material 14 forms the middle layer, and the moisture retention layer 16 forms the inner layer—adjacent the skin of the subject—of the composite material 10. However, in certain embodiment of the invention, the temperature retention material 14 may form the inner layer of the composite material 10 adjacent the skin, and the moisture retention layer 16 may form the middle layer, and the vapor barrier material 12 forming the outermost layer of the composite 10.

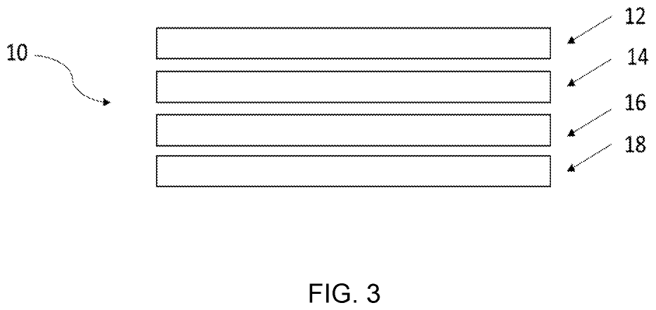

As is illustrated in FIG. 3, certain embodiments of the composite 10 may include a semipermeable material 18 such as polypropylene adjacent to or contacting the skin to wick away any unwanted moisture already present on or that may develop on the skin.

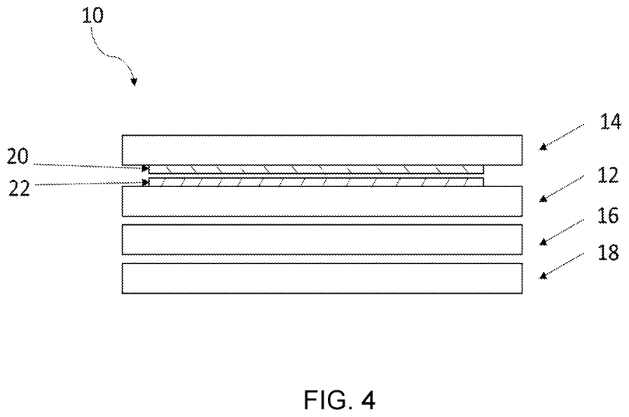

FIG. 4. Illustrates one preferred embodiment of the invention. The composite 10 includes a semipermeable polypropylene material 18 adjacent to or contacting the skin, a moisture retention material 16, and a vapor barrier material 12. A temperature retention material 14 may be removably attached to the vapor barrier material 12 through, for example, a hooks and teeth attachment means 20, 22 commonly known as Velcro®. This embodiment of the composite may be advantageous in that the temperature retention material may be easily removed, rewarmed or rechilled, and then reattached to the vapor barrier material to continue the thermal treatment. Alternatively, a user may replace the temperature retention material 14 with a different temperature retention material 14 during the course of treatment of the thermal injury. For example, treatment of the thermal injury may require alternating heating and cooling of the body or limb. In other situations, it may be advantageous to replace the heat retention material with a different heat retention material that may be heated/cooled to a specific higher or lower temperature (e.g. such as may be controlled through the use of certain PCM's) than the heat retention material that is replaced.

In still further embodiments of the invention, the composite 10 may include two or more layers of material. For example, one certain embodiment of the composite 10 may include, in order, a first vapor barrier material layer 12 adjacent to or contacting the skin, a moisture retention material layer 16, a temperature retention material layer 14, and a second vapor barrier material layer 12.

Further, it is contemplated that two or more of the materials of the composite 10 may be integrated or combinable into one layer. For example, the moisture retention material 16 and the temperature retention material 14 may be formed into a separate composite layer.

In other certain embodiments of the invention, the composite 10 may be formed into a design that is sized and shape to conform to a part of the body such as a hand, foot, arm, or leg. For example, the composite 10 may be formed into a glove, mitten, or sock to fit over a hand or foot. Alternatively, the composite material may be formed into a sleeve to fit over an arm or leg. Further embodiments of the invention may include a unitary or continuous glove and sleeve, mitten and sleeve, or sock and sleeve.

Alternatively, the composite material 10 may form a part of a larger covering or bandage, that may be used to wrap the body or limb. The composite 10 may be attached to the body or limb through known attachment means such as friction fitting, medical tape, pins, clips, clasps, hooks, Velcro®, or any combination of attachment means. Preferably, the composite may be secured to the body or limb using Velcro® for ease of use.

In other certain embodiments of the invention, the composite material 10 may be used to form individual thermal packs, similar to what are commonly known as handwarmers or ice/heat packs. The use of thermal packs for therapeutic applications, and especially heat packs, is well known. Individual thermal packs may be used to treat cold or heat related injuries by application directly to the afflicted area and/or inserted into other garments or the like configured to receive the thermal pack, such as through a sleeve or pocket, and placed on the afflicted area.

Additionally, thermal packs producing an endothermic reaction—that is, cold packs—may be useful for reducing high body temperatures, and cooling off on a hot day, or otherwise generally comforting the user, including providing relief from headaches. It is also known to use cold packs for treatment of sports injuries. For athletic purposes, cold packs typically are applied to an injured anatomical area to reduce or prevent swelling and inflammation, such as may be caused by a twisted joint, cramp, or hyper-extended muscle, or an injury resulting from a traumatic impact.

In use, the thermal packs may be inserted into a bandage, or another body or limb covering that is pre-configured with a pocket to receive the thermal pack. The thermal pack also may be applied directly to the skin and held in place by any known means such as a gauze wrap secured with Velcro®, medical tape, or the like.

The components of the composite 10 may be produced separately and assembled through the use of, for example, thermal bonding, adhesive bonding, ultrasonic bonding, or stitching. Other means of assembly are readily known by those of skill in the art. Alternatively, each layer of the composite may be constructed as a single multilayer unit followed by die cutting and a means to join the layers. The layers of the composite may be joined at any suitable location as desired. Thus, the layers may be joined at or near a single edge, or at or near a plurality of edges, or throughout the layers as desired or needed to accomplish the purpose of the present invention.

The components of the composite 10, or the fully assembled composite 10 may be chemically, mechanically, electrostatically, or otherwise treated to provide additional functional or aesthetic attributes, such as softness, stretch, sterility, absorbency, repellency, odor reduction, skin care, or the like.

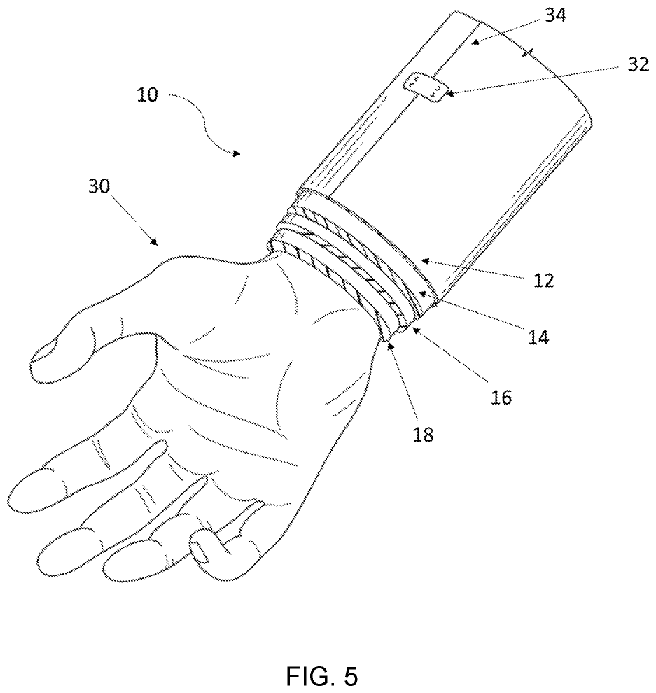

It is contemplated that the composite material 10 may be used in a medical environment, such as in a hospital or clinic, or carried by first-responders to wrap the body or a limb so that the limb may be kept warm and moist, or conversely, cool and moist. FIG. 5 illustrates the use of one embodiment of a composite of the invention. The composite 10 may be wrapped around an arm 30 of a subject in need of thermal treatment at the earliest opportunity. The composite 10 may be secured in place at a seam 34 or other suitable location using a securing means 32. As needed, the temperature retention material 14 may be activated through direct heating or cooling, or alternatively, may be manipulated to activate either an endothermic or exothermic chemical reaction that may be used to treat the subject. The moisture retention material 16—being either wetted during use or pre-wetted with a liquid (e.g. warm water)—may keep the affected area of the body or limb from drying out, to facilitate the healing process. The vapor barrier material 12 may serve to keep constant both the moisture and temperature levels surrounding the arm 30 within the composite 10. The vapor barrier material 12 may also prevent unwanted material or liquids from contaminating the composite material 10. A semipermeable layer 18 may wick away moisture from the subject's skin.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A composite of one or more materials for maintaining both moisture and temperature levels comprising:
a temperature retention material;
a moisture retention material; and
a vapor barrier material.

2. The composite of claim 1, wherein two or more of said materials are integrated to form a single layer.

3. The composite of claim 1, wherein said temperature retention material is a gel.

4. The composite of claim 3, wherein said gel includes one or more selected from the group consisting of water, a salt, cellulose, propylene glycol, and a phase change material.

5. The composite of claim 4, wherein the salt is selected form the group consisting of ammonium nitrate, ammonium chloride, calcium chloride, and magnesium sulfate.

6. The composite of claim 4, wherein said temperature retention material is one or more phase change materials.

7. The composite of claim 1, wherein said moisture retention material is one or more selected from the group consisting of a polyester, a non-woven polyester, wool, cotton, and a super absorbent polymer.

8. The composite of claim 1, wherein said vapor barrier material is one or more selected from the group consisting of a polyether laminate, a polyurethane laminate, polyethylene, vinyl membranes, polytetraflouroethylene membranes, poly lactic acid, and a coated nylon.

9. The composite of claim 1 further comprising a semipermeable material.

10. The composite of claim 9, wherein said semipermeable material is polypropylene.

11. The composite of claim 1, wherein said composite is sized and shaped to cover limb.

12. A method of treating frostbite or hypothermia comprising contacting an area of skin of a subject with said composite of claim 1.

13. The method of claim 11, wherein said composite is wetted and heated prior to contacting the area of skin of the subject.

14. A composite of one or more layers of material for maintaining both moisture and temperature levels consisting of:
a first layer including a temperature retention material;
a second layer including a moisture retention material; and
a third layer including a vapor barrier material.

15. A composite of one or more layers of material for maintaining both moisture and temperature levels comprising:
a first layer including a temperature retention material;
a second layer including a moisture retention material;
a third layer including a vapor barrier material;
said first layer having a surface and a first attachment means disposed on said surface;
said third layer having a surface and a second attachment means disposed on said surface wherein said first layer is removably attached to said third layer.

16. The composite of claim 15, wherein said first attachment means and said second attachment means includes teeth and hooks.
